(19) United States
(12) Patent Application Publication
(10) Pub. No.: US 2025/0102423 A1
(43) Pub. Date: Mar. 27, 2025

(54) SYSTEMS AND METHODS FOR CALIBRATING INSTRUMENTS THAT MEASURE TEMPERATURE AND MICROBIAL GROWTH IN BLOOD CULTURE BOTTLES

(71) Applicant: BECTON, DICKINSON AND COMPANY, Franklin Lakes, NJ (US)

(72) Inventors: James R. Petisce, Westford, MA (US); Robert Edward Armstrong, Hunt Valley, MD (US); Jingyun Zhang, Freeland, MD (US); Edward M. Skevington, Stewartstown, PA (US); Alexander W. Clark, Baltimore, MD (US); Brent Ronald Pohl, Timonium, MD (US); David J. Turner, York, PA (US)

(73) Assignee: BECTON, DICKINSON AND COMPANY, Franklin Lakes, NJ (US)

(21) Appl. No.: 18/730,824

(22) PCT Filed: Feb. 3, 2023
(86) PCT No.: PCT/US2023/012279

§ 371 Date: Jul. 22, 2024

Related U.S. Application Data
(60) Provisional application No. 63/306,797, filed on Feb. 4, 2022.

(51) Int. Cl. G01N 21/27; G01N 21/64

(52) U.S. Cl. CPC G01N 21/278; G01N 21/64

(57) ABSTRACT

A calibrator bottle and method of calibration. The calibrator bottle includes a plastic material that is doped or compounded with a fluorescent material system that fluoresces in a target range and may be used to calibrate a blood culture instrument that measures sample fluorescence to determine bottle positivity or negativity. Also described herein is a system and method to control the temperature of samples in a blood culture apparatus using a resistance temperature device (RTD) device that measures the temperature of the sample. The sample measurements are correlated with the ambient temperature to determine an ambient temperature that will provide a target bottle content temperature.

### CROSS REFERENCE TO RELATED APPLICATION

This application claims priority to and the benefit of U.S. Provisional Application 63/306,797 which was filed Feb. 4, 2022, the entirety of which is incorporated by reference herein.

### TECHNICAL FIELD

Described herein is a blood culture instrument and methods of operation, including standards for calibrating the instrument that interrogates a sample in a container by detecting a fluorescent signal and methods of operating the device to obtain an accurate temperature measurement for the blood culture.

### BACKGROUND

Fluorescence detecting apparatus is commonly used in the fields of analytical chemistry and cytometry, and for those applications, methods of calibration are well developed and well-known to those skilled in those fields. For example U.S. Pat. Nos. 5,414,258 5,093,234, 4,868,126 and many other patents and publications disclose methods of calibrating flow cytometers and fluorescence microscopes. The apparatus and method described herein is used in the field of biological sample diagnostics, in particular the detection of microbial growth in a sample bottle containing a blood sample, culture media and an indicator that reflects changes in the sample that are indicative of microbial growth. For example, such an indicator will fluoresce in response to changes in the oxygen content in the sample, or the carbon dioxide content of the sample or the pH of the sample, such changes being indicative of microbial growth in the sample.

US Patent Publication No. 20050287040 describes a fluorescent validation plate to calibrate an instrument that optically interrogates samples to observe changes in fluorescence over time. The '040 Patent Publication describes a fluorescence validation material that has a first layer selected from a fluorescent layer or a reflective layer and a second light attenuating layer that is either continuous or that has discrete attenuating and non-attenuating regions. According to the '040 Patent Publication, the advantage of using a second light attenuating layer along with the fluorescent layer or a reflective layer is that the function of the fluorescence validation material may be modified simply by varying aspects of the second light attenuating layer. In a multifunctional fluorescence validation plate, all wells may make use of the same fluorescent material with wells requiring different functions using different second material layers. According to the '040 Publication, fluorescent and reflective materials are available but are not suitable for fluorescence validation purposes because the emitted light properties (e.g., intensity, spectra, and polarization state) are not precisely controlled or, for intensity specifically, the emitted light is too bright, out of range of the detector. These materials become suitable for fluorescence validation purposes with the addition of suitable, precise, optical validation materials such as the second light attenuating layer.

Instrument calibration is necessary to provide an accurate reading from a container from a raw reading. Calibration is required to address and compensate for aspects of the system that may adversely affect the accuracy of a raw reading. It is important for operating the system accurately and effectively to understand differences in readings attributable to the sample and to differentiate those differences from difference in readings due to variations in the operation of the system in which the test is conducted.

Certain instruments (such as the BACTEC 9050 Blood Culture System that is commercially obtained from Becton Dickinson of Franklin Lakes, New Jersey) use special calibrator bottles that provide a fluorescent output that is within a certain tolerance of a target value, and has rotational stability relative to the measurement system (it does not change fluorescent output when it is rotated in the measurement station). To facilitate repeated use, calibrators require a consistent fluorescent output over a significant period of time to ensure consistency of results. Calibrators that have a less consistent fluorescent output over time, or do not maintain a high degree of consistent fluorescent output for a significant amount of time are less desirable.

The goal of the calibration process is to calculate an adjustment parameter that, when used to normalize the raw readings from a calibrator in the various sample stations (a sample station being the rack location that is interrogated by the sensor), the normalized reading is the standard value. For purposes of this analysis, the calibrator is referred to as an instrument (INST) calibrator, but the analysis is not specific to instrument calibrators.

The BACTEC 9050 has a drum-shaped rack, with bottle receptacles placed in rows around its perimeter. Each row is referred to as a ring. When the sample station (i.e., the rack receptacle that may carry the instrument calibrator to be interrogated by the sensor) and the control station (i.e., the reference receptacle carrying a specially prepared calibrator bottle) both contain calibrator bottles, the assumption is that any variation in the raw readings of the two values from the two bottles in the different receptacles will be proportional to their raw readings, and the ratio of the raw readings from the two receptacles will be constant.

In calibration mode, a normal (NORM) value is calculated with data collected during a calibration process where instrument calibrators are placed into the sample stations of the instrument. Raw readings are collected from the instrument calibrators and the prepared calibration reference in sample station 0 (sample station zero is simply a reference position in rack) of each rack, and entered into the following equation:

$$NORM = \frac{{INST}_{raw}^{st - cal} * STD}{{REF}_{raw}^{0 - cal}} . \qquad ( 1 )$$

It is important to note that, in the racks configured as a multi-row drum, the sensors are all in one column. Each sensor in the column is aligned with a row in the multi-row, multi-column rack. The sample stations 0 for each row are aligned in a single column, so that all calibration bottles/devices in sample stations 0 in each row of the rack are read at the same time.

To illustrate calibration using an instrument calibration standard, assume that a standardized calibrator value represents a reading of 0.792V on the scale of the analog to digital (A/D) in the measurement electronics. This is the measurement for the instrument calibrator bottle when measured in a sample station.

The calibrator bottles in the reference station for each rack row (station 0) of each ring are considered part of the instrument measurement system, and as such are compensated for when the NORM values are calculated with raw readings from the instrument calibrators.

The STD value is a multiplier that is included in the NORM calculation so the NORM value may be stored as an integer. Otherwise, the NORM value would be a floating point number close to unity because of the proximity in value of the instrument calibrator and REF values. When the NORM value is used to normalize a reading, it is divided by the STD value to recover the actual ratio of INST to REF.

The normalized value for a bottle in a sample station is calculated as follows:

$${VAL}_{norm}^{st} = \frac{{VAL}_{raw}^{st - read} * STD}{{REF}_{raw}^{0 - read}} * \frac{STD}{NORM} \qquad ( 2 )$$

Replacing NORM with its equation:

$${VAL}_{norm}^{st - read} = \frac{{VAL}_{raw}^{st - read} * STD}{{REF}_{raw}^{0 - read}} * \frac{STD}{\frac{{INST}_{raw}^{st - cal} * STD}{{CAL}_{raw}^{0 - cal}}} \qquad ( 3 )$$

Which reduces to:

$$= \frac{{VAL}_{raw}^{s t - read} * S T D}{{REF}_{raw}^{0 - read}} * \frac{{CAL}_{raw}^{0 - cal}}{{INST}_{raw}^{st - cal}} \qquad ( 4 )$$

Which can be rewritten as:

$${VAL}_{norm}^{st - read} = {VAL}_{raw}^{st - read} * \frac{STD}{{INST}_{raw}^{st - cal}} * \frac{{CAL}_{raw}^{0 - cal}}{{REF}_{raw}^{0 - read}} \qquad ( 5 )$$

The

$$\frac{STD}{{INST}_{raw}^{st - cal}}$$

ratio is a constant that compensates for the difference between the reading obtained from an instrument calibrator in the sample station during calibration, and the standard value. As noted above, these readings are different because of the differences in distance between the bottom of the bottle in that station and the sensor that measures it from receptacle to receptacle, differences in the light intensity of the LEDs in the sensor, etc.

The

$$\frac{{CAL}_{raw}^{0 - cal}}{{REF}_{raw}^{0 - read}}$$

ratio is calculated during the fluorescent reading of a media bottle and raw compensates the reading for any changes to the measurement system that affects both the sample station and the reference bottle in the same ring. These changes may be temperature changes that affect the LED intensity in the sensors, degradation in the sensor (e.g. LED) intensity due to aging, or a change to the position of the rotor relative to the measurement board after servicing the drum or measurement board. While instrument standards that have a high degree of stability over time are useful calibration tools, because high volume instruments like blood culture incubator/readers require multiple standards, use of an expensive set of instrument standards for calibration is expensive. Therefore, alternatives to these standards continue to be sought.

With regard to heating the bottles in the instrument to facilitate microbial growth (i.e., incubation) blood culture instruments incubate the sample bottles, which are moved past sensors (or sensors are moved past the sample bottles) to monitor indicators in the bottle for changes indicative of microbial growth. The sensors are used to interrogate the sample to ascertain whether or not microbial growth has occurred in the sample container. Users of blood culture instruments are also required to verify the temperature of the blood culture bottles by placing an independent temperature probe in the incubation chamber close to the blood culture bottles, and periodically reading the independent temperature probe manually. These quality control checks must be recorded by the user. Currently, there is no mechanism for accurately determining the temperature of a sample inside a blood culture bottle using measurements of the ambient temperature in a blood culture apparatus. Offsets between the air temperature in the housing and the temperature in the culture bottle may be determined, but those offsets may vary over time. Therefore, ways to determine the temperature of the blood culture in the sample container (i.e., the bottle) continue to be sought.

In sum, calibration of laboratory instruments is complicated. Also, since there are many system variables, the operation of the laboratory instrument must be able to adapt to changes in the internal environment of the laboratory instrument, and provide readings that are standardized and that do not vary based upon changes in operating conditions.

### BRIEF SUMMARY

Described herein are calibration devices that are formed from plastic that is doped or compounded or implanted with a dye or pigment or compound the fluoresces in a desired or predetermined wavelength range. The fluorescent dye or pigment or compound or combination of fluorescent dyes/pigments/compounds is referred to as a fluorescent material system herein. In one aspect, the plastic that contains the fluorescent material systems is one or more injection molded sheets that contain the fluorescent material system that fluoresces when excited with light. The molded sheets are provided with one or several fluorescent dyes/pigments/compounds to choose from so that their excitation and emission spectrum may be selected for the particular instrument in which the calibration devices will be used. In one aspect the fluorescent dyes are organic fluorescent dyes.

In another aspect, the organic dye that fluoresces is doped into/compounded with the plastic material to form fluorescent plastic materials that are used to form the calibrator device. In one aspect, the calibrator device takes on the shape of the containers used in the system to be calibrated. In one aspect, the calibrator device is a bottle formed from the fluorescent plastic materials.

For example, if the instrument to be calibrated is an instrument that incubates and optically interrogates blood culture bottles for indicia of microbial growth therein, then the calibrator device is shaped like a blood culture bottle so that it may be received in rack receptacles sized to receive the blood culture bottles for interrogation. The amount of dye doped into the plastic that forms the calibrator instrument bottle is a matter of design choice. The amount of dye and its wavelength range of emission is selected based on the sensors to be calibrated. One of ordinary skill in the art may select a dye with a target absorption characteristic that will yield the desired fluorescence.

In one aspect, the instrument has multiple detectors, because the sample containers (typically bottles) are detected in fly-by fashion Therefore, there is provided a column of multiple sensors, each sensor aligning with a row of the rack that holds the sample containers with sample therein. As the rack is moved, the sample containers move past the sensors (i.e., the containers “fly by” the sensors) and the sensors interrogate the sample bottles to read its fluorescence. Since the device has multiple sensors, the device requires multiple calibrators. The calibrators are required to be uniform in properties, without variation, so that any differences in sensor readings are not the result of variability in the calibration standard.

For example, in a system where the containers are stored in a rack and, in fly-by fashion, are read by a sensor, changes in distance between the containers and the sensors, for example, may lead to variations in the intensity reading from the sensors that is not due to the sample in the container (and its effect on the indicator in the container), but rather variations in the sensitivity of the system itself.

In one aspect, a sleeve may be placed over the calibration device (e.g., the bottle). Such a sleeve restricts the fluorescent output to the region of the bottle aligned with the sensor. In a blood culture instrument, the bottom of the bottle is aligned with the sensor. The sleeve may also have a feature that aligns it with the receptacle in the rack in which the bottle is received, providing the bottle with a target orientation in the receptacle. This will provide a target alignment in those aspects of the invention where the bottle receptacle has a light pipe for indicating that a receptacle is occupied by a bottle therein.

In one aspect, the bottle material is polycarbonate.

In one aspect, the instrument contains racks for receiving multiple containers of fluorescence detection. The racks have an array of rows and columns to process a significant number of containers. In one aspect, the racks are drum shaped and each row is circular. The sensors are positioned in a column adjacent the exterior of the drum. Each sensor in the column of sensors is aligned with a row in the rack so that one sensor is positioned to detect fluorescence of each bottle in the row as it rotates past the sensor (i.e., the fly-by arrangement described above).

For calibration, the rack has one column that is designated to receive reference calibrator devices (e.g., calibrator bottles). Calibrator bottles are placed in the other columns. The sensors read the raw fluorescent values from the calibrators and the reference bottles. Those values are then used to evaluate the accuracy of the instrument in reading the fluorescence of the blood culture bottles.

Significant deviations in the readings of the calibrator bottles from the expected values may indicate that the drum and the sensors are not in their appropriate relative positions. In remediation, the relative position of the drum and sensors is adjusted to achieve the target readings from the calibrator bottles. When in-range readings for at least some calibrator bottles are obtained, bottles with out of range readings are replaced and the calibration process is repeated until all readings of the calibrator bottles are in range.

In another aspect, described herein is a method of calibrating an instrument for detecting sample fluorescence. According to the method, a calibration device is provided. The calibration device includes a plastic material, wherein the plastic material is doped or compounded with or implanted with a fluorescent material system that has at least one a dye or pigment or compound that has a fluorescent emission spectrum within a first predetermined wavelength range when excited by light of a second predetermined wavelength range. The calibration device is placed in an instrument with a sensor. The calibration device is then brought into alignment with the sensor, after which light is directed from a light source, wherein the light source emits light in the second predetermined wavelength range to produce fluorescence in the first predetermined wavelength range. Subsequently it is determined if the sensor detects the fluorescence in the first predetermined wavelength range.

Also described herein is a temperature sensor such as a resistance temperature detector (RTD) disposed in an instrument for processing biological samples, such as blood culture samples. Such instruments require temperature control to ensure that the biological samples are incubated at the correct temperature. 24.

In one aspect, described herein is an instrument for blood culture incubation The instrument has a housing, a rack disposed in the housing. The rack having plurality of rack receptacles for receiving a plurality of blood culture sample containers. At least one rack receptacle is configured to receive a temperature measuring device. The temperature measuring device may have a resistance temperature detector. In one aspect, the instrument is configured to read the temperature recorded by the resistance temperature detector and, from that temperature infer the temperature of a blood culture in a sample container in the instrument. In a further aspect the instrument may have a controller. The controller, when receiving a temperature from the temperature measuring device, compares such temperature to a set temperature, and, based upon the comparison, controls the temperature in the housing.

In one aspect, the rack receptacles may have a light pipe and the receptacle configured to receive a temperature measuring device has the temperature embedded in the light pipe. In a further aspect, the receptacle configured to receive the temperature measuring device may have a thermal grease or a thermal conductive pad applied thereon. In a further aspect, the resistance temperature detector may be electrically coupled to a cable comprising a power wire, a ground wire, and a serial communication wire. In a further aspect, the cable may be coupled to a slip connector.

In a further aspect, the temperature measuring device is container-shaped, wherein the shape is configured to be received by the receptacle configured to receive the temperature measuring device. According to this aspect, the resistance temperature detector is immersed in liquid inside the container-shaped measuring device and may be coupled to a cable with contacts that are configured to electrically connect to a slip connector adjacent to the receptacle configured to receive the temperature measuring device. In one aspect, the contacts are contact rings.

In another aspect, the contacts are placed in spaced relation on the surface of the temperature measuring device. In yet a further aspect, the resistance temperature detector is coupled to a cable comprising a contact strip, with contacts that are configured to electrically connect to corresponding spring contacts in electrical communication with a microprocessor. Optionally, the resistance temperature device further comprises a transmitter and a power source and is rechargeable.

### BRIEF DESCRIPTION OF DRAWINGS

FIG. 1 illustrates emission spectrum for different doped plastics.

FIG. 2 is a calibrator device assembly according to one aspect herein;

FIG. 3 is an end view of the calibrator device assembly of FIG. 2;

FIG. 4 is a side view of the assembled calibrator device assembly of FIG. 2;

FIG. 5 is an illustration of a blood culture bottle;

FIG. 6A is a comparison of the fly-by readings obtained from a plastic bottle with a sensor as calibrator and a plastic bottle doped with fluorescent dyes;

FIG. 6B illustrates the transmittance of neutral density filters used to obtain the readings in FIG. 6A;

FIG. 7 illustrates fluorescent emission spectra for different bottle materials doped with dye demonstrating feasibility of using bottles doped with dye as calibrator bottles;

FIG. 8 is a top down view of the interior of an incubator with a circular rack for receiving blood culture bottles;

FIG. 9 is a rack receptacle with a temperature probe;

FIG. 10 is an exploded view of a bottle and receptacle therefore;

FIG. 11 is a side view of a slip connection on the light pipe rack receptacle;

FIG. 12A is a phantom view of a blood culture bottle with an RTD device immersed in fluid in the bottle;

FIG. 12B is a neck end view of the blood culture bottle illustrated in FIG. 12A;

FIG. 13 is a schematic of an illustrative hard wire connection to an RTD device;

FIG. 14A is a phantom view of an RTD device disposed in a bottle in accordance with a different aspect;

FIG. 14B is a neck end view of the blood culture bottle illustrated in FIG. 14A;

FIG. 15 is a schematic view of a docking station for a bottle-shaped RTD device;

FIG. 16 illustrates a bottle with a temperature sensor disposed therein to provide a temperature of the solution in the bottle;

FIG. 17 is a side view of a calibration bottle with an RTD device configured to communicate with and RTD reader according to an alternative aspect of the devices described herein; and

FIG. 18 illustrates an aspect of an RTD reader that uses gripper fingers.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Currently, the BACTEC™ instrument is calibrated using specially prepared BACTEC™ calibrator glass bottles. These calibrator glass bottles contain a multi-component chemical formulation which emit a fluorescence signal at a specific wavelength and emission intensity. Calibrator bottles are produced separately from BACTEC™ sensor bottles and require dedicated production runs and storage areas once they are produced.

Described herein are calibrator devices that deploy plastic that is impregnated or compounded or mixed or doped with one or more dyes and/or one or more pigments that fluoresce in response to an external light source. The one or more dyes and/or one or more pigments is referred to as a fluorescent material system herein. The calibrator devices, in one aspect, are molded from the doped/compounded plastic material. In other aspects, the calibrator devices receive a doped plastic component that will fluoresce when stimulated by an external light source. In one aspect, the instrument being calibrated is a blood culture instrument. Such instruments have racks with a plurality of receptacles. Each receptacle is sized to receive a blood culture container (“bottle” hereinafter). During instrument calibration, the receptacles receive the calibrator devices. Therefore, calibrator devices have a dimension and a configuration that allows them to be received by a rack receptacle. In one aspect, the calibrator bottles are shaped like the bottles received by the racks. The bottle material itself is used to calibrate the instrument, thereby obviating the need for calibrator bottles that carry a specially made liquid standard inside that may be used for calibration. The calibrator bottles retain their fluorescent characteristics (i.e., their fluorescent spectrum response to light) to for a significant period of time.

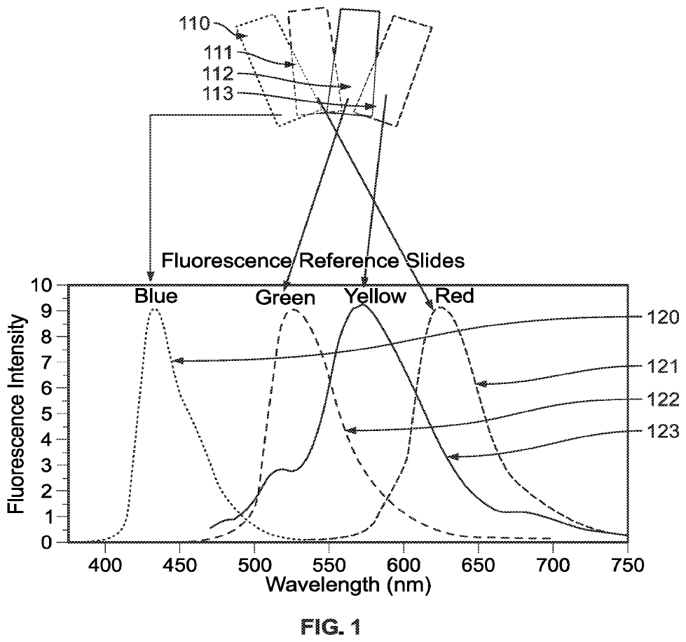

Referring to FIG. 1, illustrated are injection molded sheets, each of which contains fluorescent material system that has at least one dye that fluoresces when excited by a certain wavelength of light (i.e., the excitation wavelength). Illustrated in FIG. 1 are sheets 110, 111, 112, and 113 which are doped with different organic dyes that will cause the doped plastic to fluoresce in the blue, red, green, and yellow ranges, respectively. As illustrated in FIG. 1, the wavelength ranges are about 400 nm to about 500 nm (the blue range, 120), 550 nm to about 750 nm (the red range, 121), about 450 nm to about 650 nm (the green range 122), and about 475 nm to about 750 nm (the yellow range). While there is wavelength overlap in the wide ranges, the peak wavelength ranges for each wavelength illustrated in FIG. 1 do not overlap. Conventional dyes are used as dopants for the plastic sheets. For example, to fluoresce in the red spectrum, the plastic sheet is doped with Texas Red dye (rhodamine dye). One skilled in the art is aware that rhodamine dyes are actually a family of dyes that possess absorption and emission spectra in the visible—near infrared (NIR) region of the spectrum. One of skill in the art will be able to select a fluorescent material system that will serve to calibrate the sensors of the instruments being calculated. One of ordinary skill understands that absorption and fluorescent spectroscopic properties are a function of the molecular properties of a specific dye. See Maeda, H. et al. Absorption and Fluorescence Spectroscopic Properties of 1- and 1,4-Silyl-Substituted Napthalene Derivatives, Molecules 17, pp 5108-5125 (2012), which is incorporated by reference herein. Dyes and dye loadings for fluorescence in a particular spectrum are well known to one skilled in the art and not described in detail herein.

For example, if one wished to use a fluorescent material system having fluorescence emission spectrum at about 360 nm when excited using a light source with a maximum excitation wavelength of about 300 nm, one would review a list of potential dyes/dopants/pigment and ascertain which dye/dopant/pigment or combination of dyes/dopants/pigment that would provide a fluorescent material system with the target excitation and emission spectra. For example, referring to Table 1 below, naphthalene or p-terphenyl are potentially suitable because the characteristic fluorescence emission spectral range for either includes 360 nm and their peak excitation wavelengths are very close to 300 nm.

Subsequently, the concentration of fluorescent compound or fluorescent material system is controlled to provide a desired fluorescence emission intensity. One skilled in the art may evaluate different concentrations of the fluorescent compound or different concentrations of the components of a fluorescent material system that will provide the desired fluorescence emission intensity. In a different aspect, the concentration of the fluorescent dye or different concentrations of the components of a fluorescent material system is chosen by utilizing the fluorescence quantum yield value for the candidate fluorescent material.

For example:

$$Quantum Yield = Molar concentration of fluorescence emission / Molar concentration of the fluorescent compound added into the plastic material \qquad ( 6 )$$

Fluorescence quantum yields of compounds are reported in published books or scientific journal articles such as Berlman, I., “Handbook of Fluorescent Spectra of Aromatic Molecules” (1971). As reported in Berlman, the fluorescence quantum yields for naphthalene and p-terphenyl are 0.23 and 0.93, respectively.

To provide a fluorescent system having a fluorescence emission around 475 nm when excited using a light source with a maximum excitation wavelength of 350 nm, consulting Table 1, below, a suitable candidate fluorescent compound may be one of ovalene or tetraphenylbutadiene because the characteristic excitation and fluorescence emission spectral ranges for either includes 475 nm and their peak excitation wavelengths are very close to 350 nm.

Once the fluorescent compound or combination of fluorescent dyes/pigments are selected, the concentration of the fluorescent compound or combination of fluorescent dyes/pigments are selected to provide a target fluorescence emission intensity. One of ordinary skill may select a concentration by evaluating formulations having different concentrations of the fluorescent components. In another aspect, the concentration of the fluorescent compound or combination of fluorescent dyes/pigments by utilizing the fluorescence quantum yield value for the candidate fluorescent material. A summary of fluorescent excitation and emission data is provided in the Fluorophore Reference Guide from Bio Rad which was downloaded from the internet (www.bio-rad.com/webroot/web/pdf/lsr/literature/Bulletin_2421.pdf) on Jan. 30, 2022.

One skilled in the art is aware that fluorescent intensity is also a function of molar concentration. For fluorescent measurements, the molar concentrations are typically low, such as those reported in Table 1 below. See Fonin, A., et al., “Fluorescence of Dyes in Solutions with High Absorbance Inner Filter Effect Correction”, PLoS One 9(7) (2014).

Examples of suitable dyes and their concentration and emission ranges are set forth in the table below:

TABLE 1

| | | Peak | | Peak | |
|---|---|---|---|---|---|
| | Approximate | Excitation | Excitation | Emission | Emission |
| | Molar | Wavelength | Range | Wavelength | Range |
| Dye/Dopant | Concentration | (nm) | (nm) | (nm) | (nm) |
| Anthracene | 1 × 10⁻⁵ | 360 | 260-320 | 402 | 370-470 |
| Napthalene | 6 × 10⁻⁵ | 290 | 290-390 | 330 | 310-400 |
| Ovalene | 2 × 10⁻⁷ | 342 | 300-460 | 482 | 450-570 |
| p-Terphenyl | 5 × 10⁻⁷ | 295 | 270-350 | 338 | 310-410 |
| Tetraphenylbutadiene | 3 × 10⁻⁷ | 348 | 290-390 | 422 | 370-540 |
| Rhodamine (Red) | 2 × 10⁻⁷ | 562 | 500-600 | 573 | 530-630 |

The calibrators described herein have the fluorophore dye or pigment or the fluorophore material system embedded in plastic. As such, the dye or pigments are more stable for a longer period of time than if incorporated into a liquid standard.

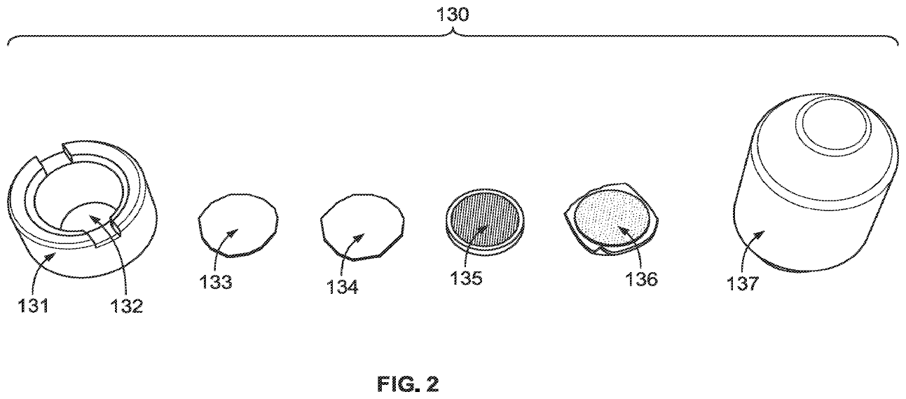

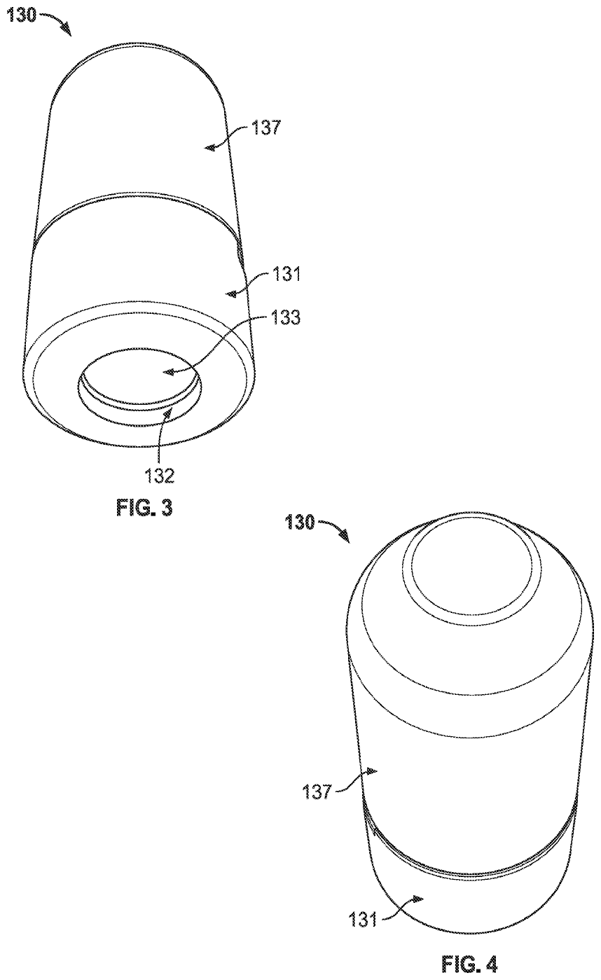

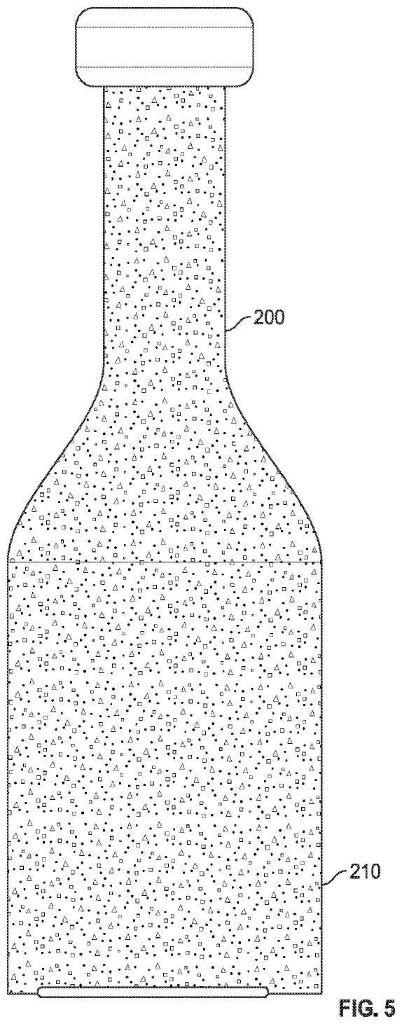

As noted above, in one aspect, the calibrator may include a plastic component which has been doped with or compounded to include a fluorescent dye or pigment. Referring to FIG. 2, a calibrator device 130 is assembled from such molded components. The molded components illustrated are a base 131, with an opening 132 therein. Inserted in the opening are, in order from the opening, a neutral density filter 133, the fluorescent plastic 134, a backing plate 135, a foam holder 136, and an assembly top 137. The fluorescent plastic may be a thermoplastic material such as polycarbonate, for example, doped with or compounded with a fluorescent compound, for example a fluorescent dye such as one or more of the fluorescent dyes described above. The thermoplastic material is selected based upon its stability (oxidative, hydrolytic and photochemical). Additionally, the thermoplastic material is selected so that its inherent light absorption or emission characteristics do not overlap or compete with the absorption and emission ranges of the fluorescent compound or the fluorescent material system. Additionally, the fluorescent compound may also be a fluorescent pigment. FIG. 3 is a bottom side view of the calibrator device with the neutral density filter 133 visible through the opening 132. FIG. 4 is a side view of the calibrator device 130.

One of ordinary skill is aware that neutral density filters are used to reduce or modify the intensity of all wavelengths. A neutral density filter is used in this calibrator device because the intensity of the fluorescent signal will be too great if not modulated.

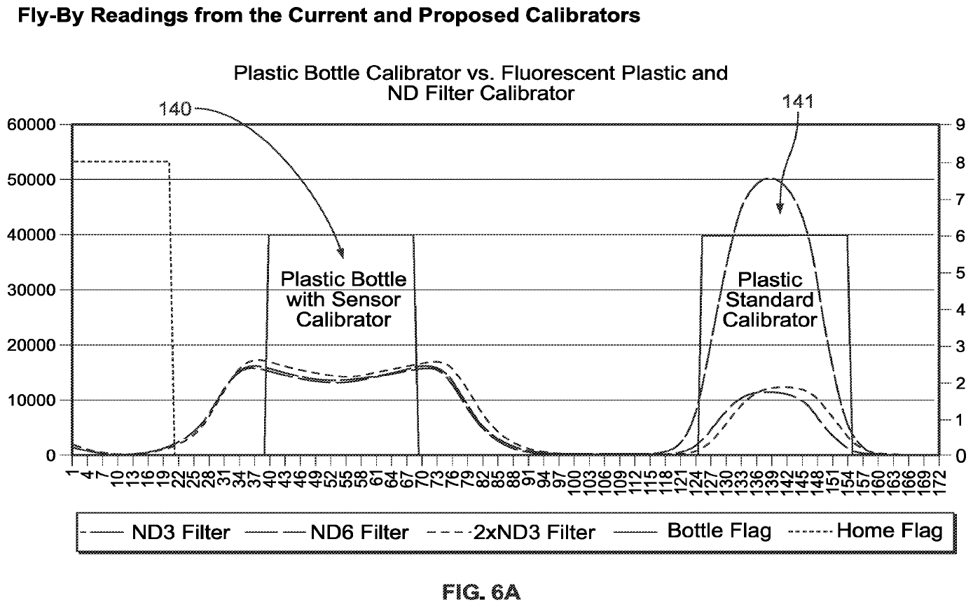

In one aspect, the calibrator device is a polycarbonate bottle formed from a plastic material doped with at least one dye/pigment or the fluorescent material system, which are the fluorescent plastic materials described above. In this aspect, a neutral density filter is not required. Such a bottle 200 is illustrated in FIG. 5. Previously, calibrator bottles have been provided where a calibration substance has been fixed in the bottom 210 of the bottle 200. The problem with such previous approaches is that the calibration substance was not sufficiently stable over time, requiring frequent replacement. Other plastics that may be doped with one or more dye(s)/pigments/compounds to be used as calibration devices as contemplated herein include nylon 6 and polymethyl methacrylate (PMMA). The solid polymethyl methacrylate matrix provides a stable environment for the fluorescent compounds. The dyes/dopants incorporated are not in solvent form, so there is no evaporation associated with their incorporation into the matrix. As such, the dyes are stable in the polymer matrix into with they are incorporated.

In practice, excitation of a calibrator bottle made from a plastic material doped with a fluorescent material system illustrated in FIG. 5 may not only excite the area of the bottle directly impinged upon by the excitation light, but may also excite other areas of the bottle when the excitation light transmits through the plastic material doped with the fluorescent material system. Consequently, the resultant fluorescence emission may be a combination of fluorescence from the area upon which the excitation light directly impinges (i.e., the primary transmission) and the fluorescence from other bottle areas impinged upon by excitation light transmitted through the plastic bottle material doped with the fluorescent material system (i.e., the secondary transmission). In mitigation, it may be desirable to control the measured fluorescence emission by eliminating any transmission of excitation light through the plastic bottle doped with the fluorescent material system so that the only fluorescence is from the primary transmission. A simple way to avoid the unwanted secondary transmission from the calibrator bottle is to coat the interior of the plastic bottle with a material system layer that blocks the excitation light transmission that would result in secondary transmission. The material system layer may contain a pigment or dye system or combination thereof that blocks the excitation light transmission. Alternatively, the material system layer may be a highly reflective material. In addition to blocking the transmittance of excitation light through the bottle thickness, it is expected that the coating on the interior of the bottle should be capable of blocking any transmission of fluorescence from the bottle area exposed to the excitation light (other than the primary transmission).

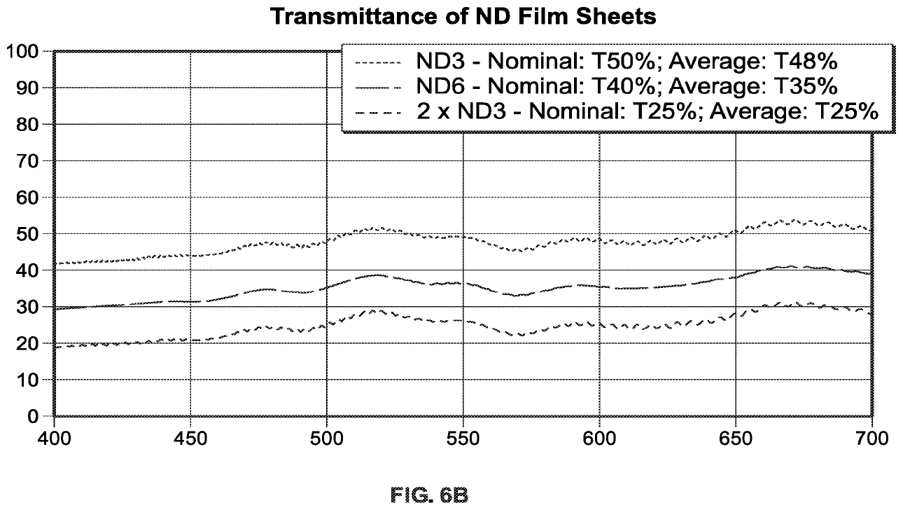

FIG. 6A provides a comparison in readings using two different calibrators to demonstrate the efficacy of a neutral density filter. Since neutral density filters filter light, they are typically formed as a film or other semi-transmissive material. Plastic bottles were placed in a drum shaped rack as described above. Three types of neutral density (ND) filter systems were evaluated. The blue curve was obtained using an ND3 filter that has a nominal transmittance of about 50%. The orange curve was obtained using an ND6 filter that has a nominal transmittance of about 40%. The gray curve was obtained using a 2×ND3 filter (i.e. two ND3 filters placed one on top of the other) that have a nominal transmittance of about 50% each and a collective nominal transmission of about 25%. FIG. 6B illustrates the transmission of the neutral density filters that were used to obtain the data in FIG. 6A over a range of wavelengths. There is a slight increase in average transmission with increasing wavelength.

These filters or combination of two filters were placed in the calibrator device illustrated in FIG. 3 having the components as illustrated in FIG. 2. The readings 140 on the left are for a prior art calibrator 140 that has a sensor calibrator (such as a cured liquid formulation described above) disposed in the container. The readings 141 on the right were obtained using the calibrator device 130 illustrated in FIGS. 3 and 4. Spectra readings 141 demonstrate that the fluorescence of the device may be adjusted/modulated by adjusting the amount of incident light exciting the doped plastic component 134 using different neutral density filters. Comparing the emission spectra between 140 and 141, the defined peaks in 141 provide a higher quality signal that the signals from the prior art calibrator.

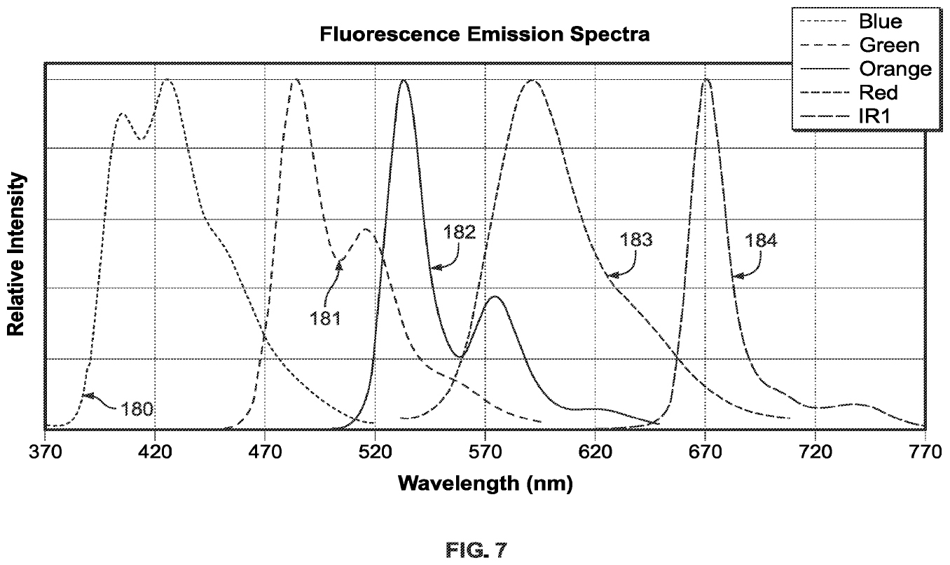

FIG. 7 illustrates spectra for a calibration system that uses a set of fluorescent plastic materials with different emission spectra. Therefore the set of fluorescent plastic materials from which the emission spectra illustrated in FIG. 6 were obtained cover a broad spectral range from about 260 nm to about 600 nm and an emission range from about 370 nm to about 630 nm. The wide spectral range allows the user to select, from the set of calibrators, the calibrator with spectral properties that most closely correspond to the spectral properties of the analyte to be interrogated by the sensor. This allows the calibrator to be interrogated by the sensor without making adjustments to the apparatus (e.g., changing parameters such as the width of the slit through which the sensor signal is projected onto the calibrator device, or a change in wavelength settings from those settings used to read the samples.

Calibration devices described herein are used to calibrate the sensors in the instrument during a calibration cycle (i.e., during system set up) and also calibrate the sensors during a test cycle (i.e. when the system is in use). The amplitude of the reading from the calibration device in the calibration receptacles in the rack (i.e., station 0) is constant over some period of time, but it may be high enough on the scale of the analog to digital converter to minimize the signal/noise ratio. It is noted that the same calibration device may be used for calibration in the test cycle and calibration in the calibration cycle. The ratio of the reading obtained from the calibration device during a test cycle to the reading from the same calibration device during the calibration procedure is the same, irrespective of the absolute fluorescent output from reading of the calibration device. The calibration devices used in the calibration station (also referred to as station 0) of each row of receptacles (i.e. each rack row) in the incubator three rings do not have to be expensive to manufacture to be reliable and provide good results over time. As such, the fluorescent plastic materials described herein provide an economical alternative to other calibration devices used to calibrate systems such as the BACTEC that optically interrogate samples to detect fluorescence indicative of a positive blood culture.

As noted above, the system described herein is calibrated initially and during use. In one aspect, the calibration devices used to calibrate the system initially are different from the calibration devices that are part of the instrument and used for day to day operations. Also, the calibration device that are used to calibrate the system initially are used to calibrate the calibration devices that are part of the instrument.

### Temperature Calibration/Control

Blood culture instruments are designed to keep the media and blood in the blood culture bottles being processed close to a specific temperature setpoint as configured by the user. To do this, the blood culture bottles are processed in an incubation chamber, and the air in the chamber is controlled to the configured temperature using a heater and blower to transfer heat into the incubation chamber. A control temperature probe is used to measure the amount of heat transferred into the chamber.

The temperature control probe used to control the incubation system is typically placed anywhere in the heated air path through the incubation chamber. Generally, the control temperature probe is located just after the heater in the air path. In this location the control probe measures the highest temperature of the air in the air path. As the heated air follows the air path through the incubation chamber it loses heat to the bottles and other components in the incubation chamber before returning to the blower and heater at its lowest temperature.

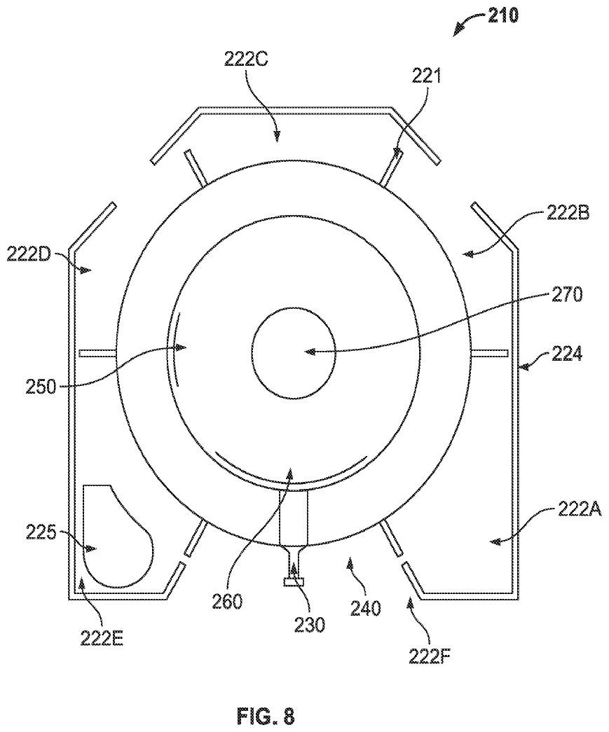

Referring to FIG. 8, there is illustrated a top down view of an example of a rotating bottle rack for blood culture incubation. The rotating bottle rack is a drum shaped rack 240 placed in a housing 224. The module 200 also includes a blower and heater 225 for keeping the bottles 230 warm. The drum-shaped rack 240 has receptacles that hold the culture bottles 230. Positioned in the interior of the housing are measurement electronics 250 and culture bottle/indicator electronics 260. Such electronics may be placed in the interior of the drum (as illustrated) or along the exterior of the drum (i.e., placed between the drum-shaped rack 240 and the housing 224). A drive motor 270 is provided to rotate the drum-shaped rack 240. As illustrated, the drum 240 housing 224 has six panels 221 that define six drum sectors (222A-222F). As illustrated, about one-sixth of the drum contents (assuming the drum is full) are available for access at any given time since the span of one sector is about the same as the span of the opening in the housing through which bottles are added to or removed from the drum 240. The culture bottles 230 may be disposed neck-inward or neck outward. In FIG. 7, the bottles are placed neck outward, so the sensors and such are located in the interior of the drum-shaped rack 240. If the bottles are placed neck inward, then the sensors and such are located along the exterior of the drum-shaped rack 240. The motor 270 is a direct drive motor that provides high torque, little to no hysteresis, low noise, reliability and simplicity.

The air path taken by the heated air in a blood culture incubator is around the blood culture bottles contained in the drum-shaped rack in the incubation chamber. The temperature of the air as it passes around the blood culture bottles is somewhere between the high temperature of the air as it exits the heater, and the low temperature of the air as it completes the air path and enters the blower.

The contents of the blood culture bottles are generally not at the temperature reported by the control probe placed in the housing. The contents of the blood culture bottle are generally lower than the temperature reported by the control probe. To adjust, an offset is added to the configured temperature setpoint, increasing the temperature at the control probe, to raise the average bottle temperature to the target or setpoint temperature. The offset is the difference between the temperature measured by the control probe and the temperature of the blood culture bottles when the system is in a steady state. For instance, in a system that targets a bottle temperature of 35° C., if at steady state the control probe is reading 35° C. and the blood culture bottles are at 34.5° C., an offset of 0.5° C. would be established for the incubation system. The heater is then controlled so that the control probe measured an air temperature of the setpoint plus the offset (35° C.+0.5° C.=35.5° C.) and the blood culture bottles would then reach the configured setpoint temperature of 35° C. because the set temperature of the probe was the needed 0.5° C. higher.

The offset required by the incubation system is dependent on several factors such as the location of the control probe in the air path, the rate that heat is lost from the incubation chamber at each point in the air path, the location of the blood culture bottles in the air path, the ambient temperature around the incubation chamber, and many other factors. The offset is determined empirically during development of the system by measuring the temperature difference between the control probe and the contents of the blood culture bottles at extremes of the operating environments in which the system is intended to work. An offset is then selected to accommodate all conditions, yet keeping the blood culture bottles within their specified range of temperature.

During system operation, operators of the blood culture instruments are required to verify the temperature of the blood culture bottles by placing an independent temperature probe in the incubation chamber close to the blood culture bottles, and periodically read the independent temperature probe manually. These quality control checks must be recorded by the operator.

As an alternative to using a universal offset value for controlling the temperature of the contents of a blood culture bottle, disclosed herein is a method and apparatus that ascertains the temperature offset between the probe temperature measurement and the temperature of the contents of the blood culture bottle using a resistance temperature detector. The method and apparatus disclosed herein also permits adjustment of the incubation offset at runtime to adjust for changes in ambient operating conditions. As used herein, ambient is the environment in the housing of the apparatus.

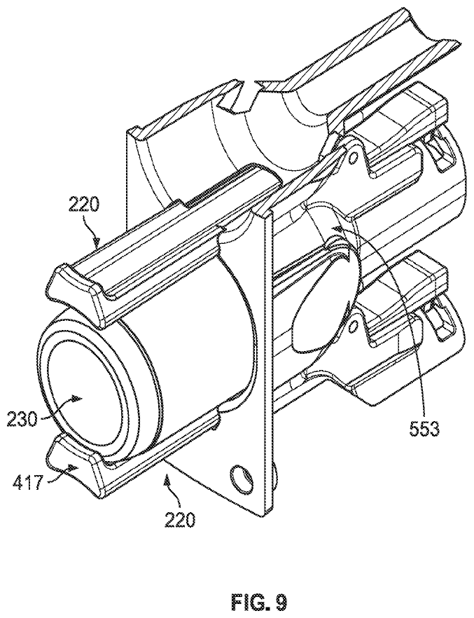

Referring to FIG. 9, a temperature probe is included in a light pipe receptacle 417 of the bottle holders 220 in the drum that hold the reference bottles 230. Assuming that the drum-shaped rack has 10 rows, there is a column of 10 reference bottles placed in each drum arranged in a single column of the drum. The reference temperature bottles, like the calibration devices described above, are placed in a single column, each receptacle being station 0 for its respective row. The reference temperature bottle 230 may also contain a piece of plastic doped with a fluorescent dye/pigment/compound with neutral density filter as described above or be formed from the plastic material doped/compounded/implanted with a fluorescent material system as described above. In an alternative aspect, the reference temperature bottle may be an existing empty bottle with no indicator dye sensor.

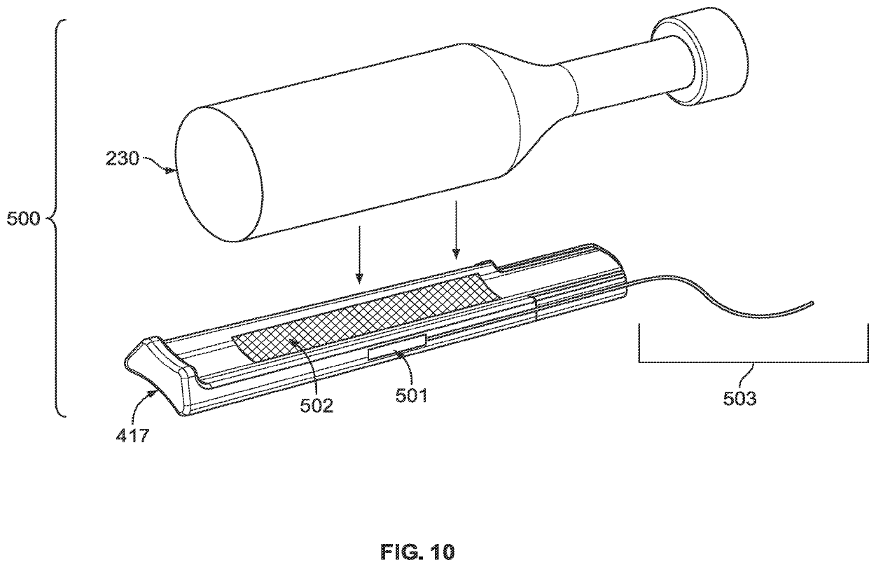

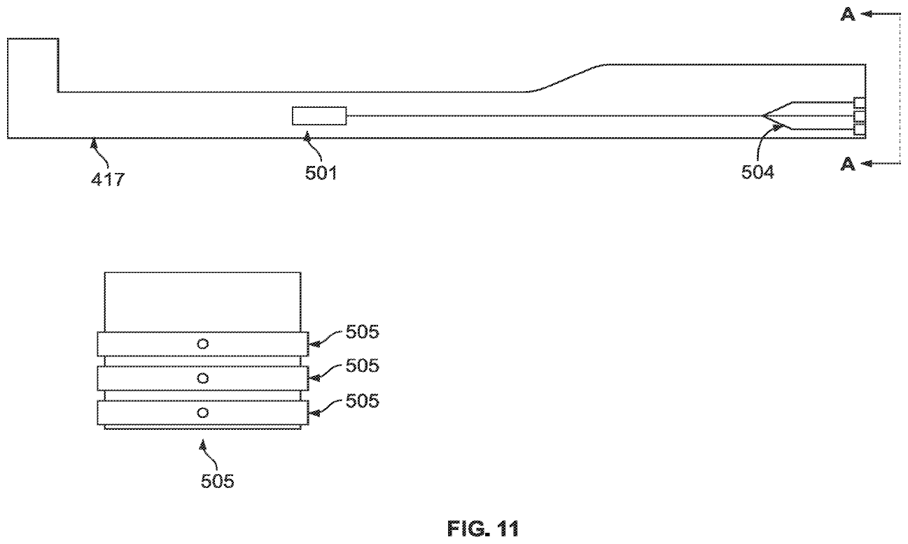

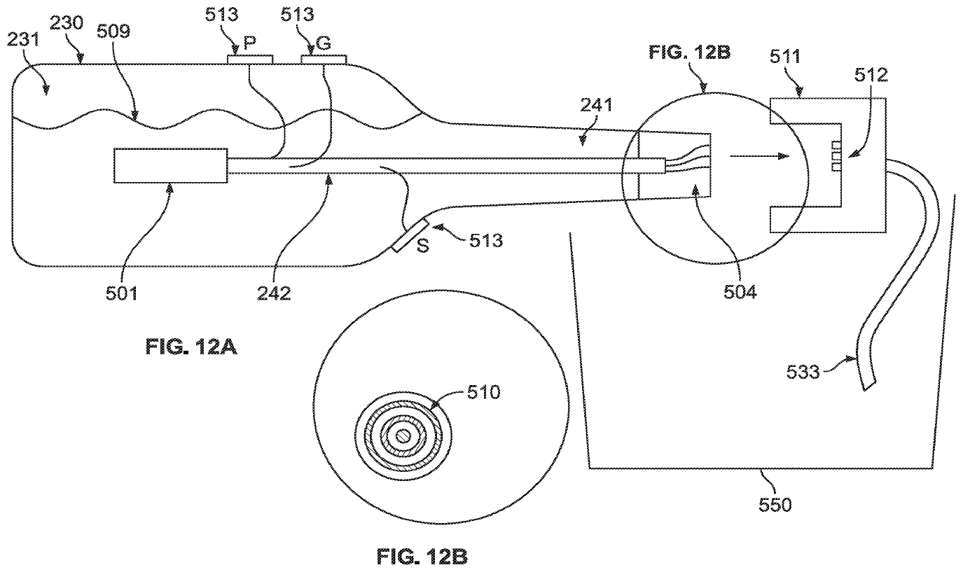

Referring to FIG. 10 and FIG. 11, there is an exploded assembly 500 of bottle 230 that is received by light pipe 417. Light pipe 417 indicates the state of the bottle in the receptacle (e.g., positive, negative, in need of resolution). As illustrated, the light pipe has a resistance temperature detector (RTD) embedded therein that is in direct contact with the reference bottle 230 when the reference bottle is set on the light pipe 417. RTDs are well known to one skilled in the art and are not described in detail herein. A thermal grease 502 or other conductive material is placed between the RTD in the light pipe receptacle 417 and the bottle 230 and may improve the heat transfer efficiency from the bottle to the RTD. The end of the light pipe receptacle 417 has wires that lead to a slip connector elsewhere in the instrument (503). Three wires 504 may be connected to wires leading to the embedded RTD in light pipe receptacle 417. In one aspect, power, ground, and serial communication contact strips 505 are attached to the wires (end view of light pipe 417). Referring to FIG. 12A, a connection 533 runs between the slip connector contacts 512 in the RTD receptacle and the controlling microprocessor electronics interface.

Referring to FIG. 12A and FIG. 12B, in an alternative aspect, the reference bottle 230 could hold a hermetically sealed volume of liquid 509 in its interior 231 in which an RTD 501 is immersed. The cable tail 504 of the RTD (with the three wires described above) may be sealed as it exits the reference bottle 230. As illustrated in FIG. 12B, which is an end view of the bottle 230 illustrated in FIG. 12A, the three wires (power (P), ground (G), and serial communications (S)) are formed as contact rings 510 to be received in complementary receptacles 512 in the cap slip connector 511. The slip connector 511 is formed as a cap into which the neck 241 of the bottle-shaped vessel 230 may be placed. The contacts in the slip connector 550 may be any conventional contact such as pogo pins, leaf contacts, etc.) In an alternative aspect, the contacts 513 (P), (G), and (S) are formed on the bottle and connected to the cable 242.

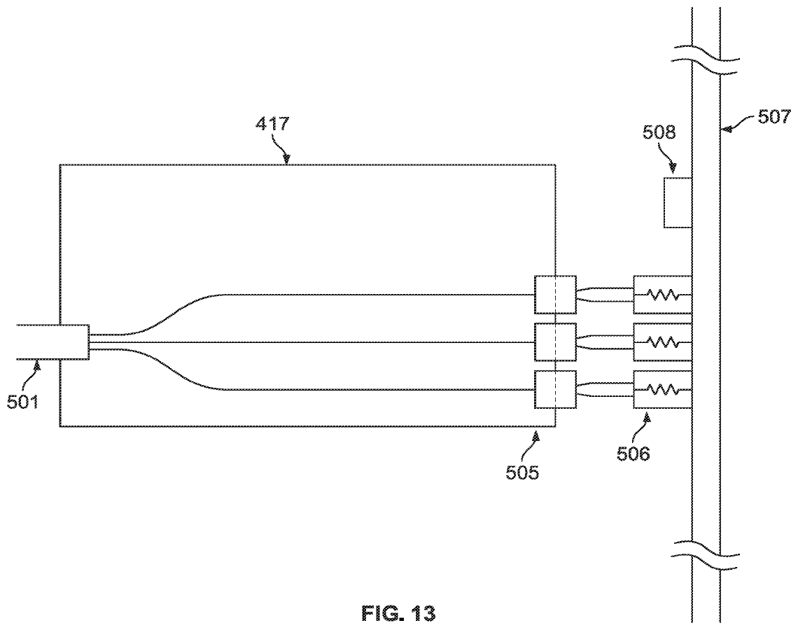

FIG. 13 illustrates the cable from the RTD 501 embedded in or placed on light pipe 417. At the end of the light pipe are placed contact strips 505 that may be electrically connected to spring contacts 506. As previously described there are three wires and three contacts for wires power (P), ground (G), and serial communications (S). Contact 506 may be mounted on a circuit board 507. LEDs 508 are provided for illuminating the light pipes.

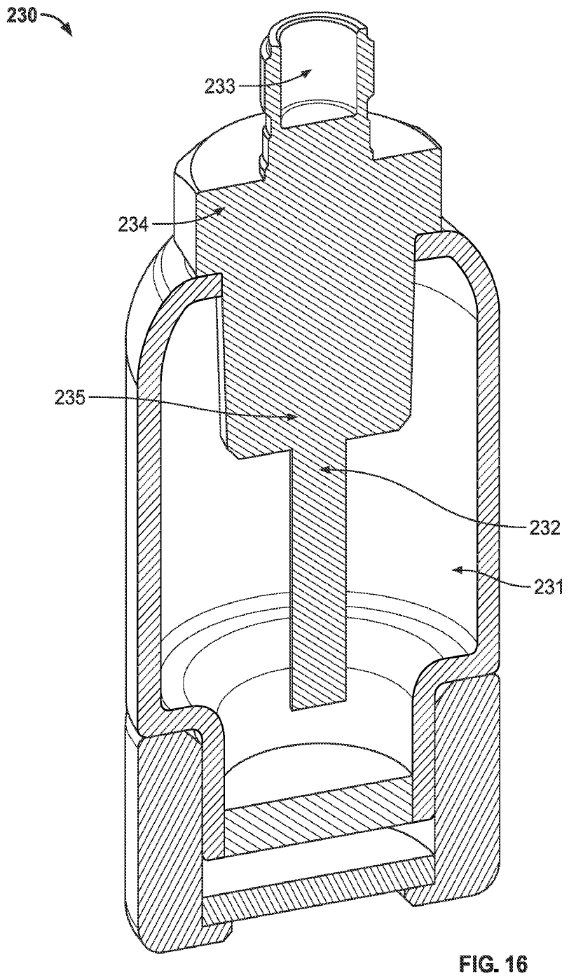

Referring to FIG. 16, the RTD device 235 may have a hermetically sealed connector 234 on one face of the reference bottle 230. In an alternative aspect, the cable tail 233 may be connected to the light pipe 417 interface described above. The cable tail 233 may also be connected to the slip connector 511. The RTD has an extension 232 so that it reaches almost to the bottom of the bottle interior 231 to ensure that portion of the RTD device is disposed in the liquid, as described elsewhere herein. The RTD is shown in cross-section, with the cut surfaces darkened.

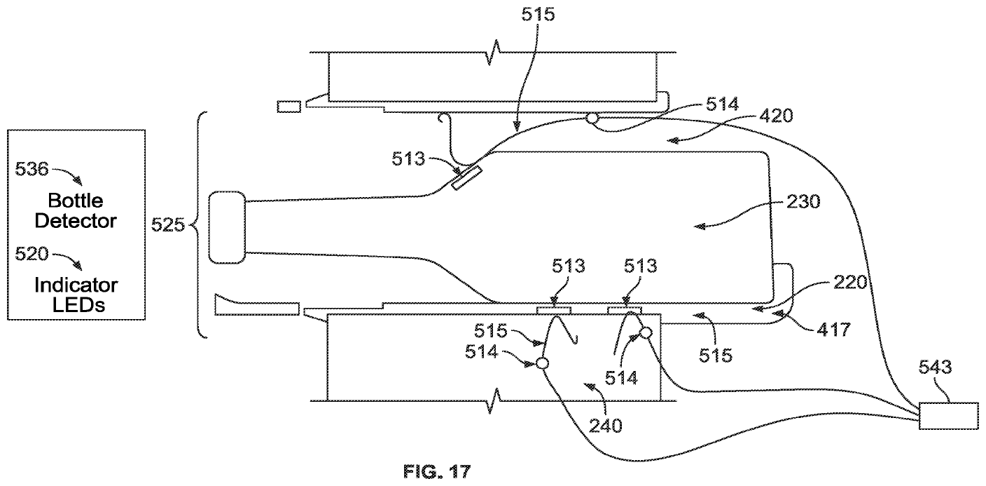

Referring to FIG. 17, in an alternative aspect, described herein is a bottle-shaped RTD reader that has wire connections 514 to contacts 513 via spring loaded conductors 515. The bottle detector 536 and the indicator LEDs 520 are placed at the neck-end 525 of the bottle-shaped RTD device 230. As described above, the bottle-shaped receptacle does contain fluid into which the RTD device is placed. The spring-loaded connections 515 are forced into contact with the bottle contacts 513 when the bottle 230 is received by the rack receptacle 240. There are three contacts 513 illustrated that connect to the RTD in the reference bottle holder 220 (for holding the proximal end of the bottle 230) with light pipe 417, along with contacts for power, ground, and serial communications. The wires 514 are also connected to the microprocessor 543 or other controlling electronics.

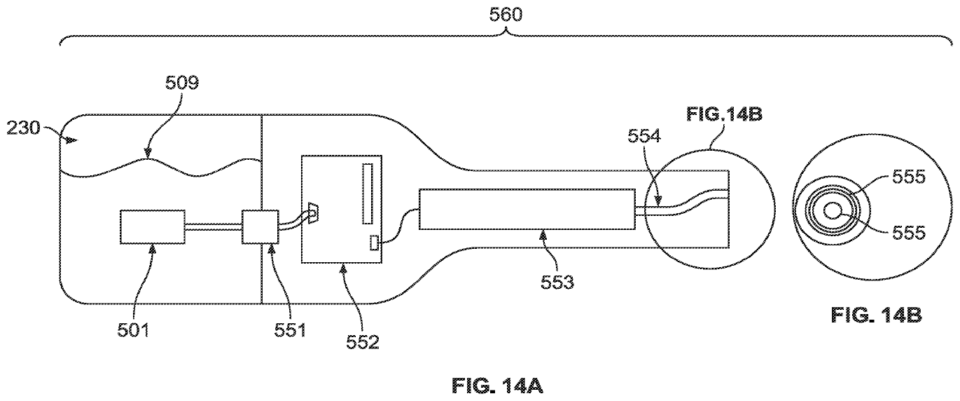

Referring to FIG. 14A and FIG. 14B, there is illustrated a rechargeable RTD device 501 disposed in liquid 509 in the bottle shaped receptacle 230. The bottle 230 has a seal 551 to keep the liquid in the portion of the bottle 230 where the RTD 501 is located. Also placed on the dry side of the bottle 230 is electronics 552 for the RTD reader with power source 560 illustrated in FIG. 14A. Examples of suitable electronics 552 includes a circuit board memory and or a transmitter with an antenna. The RTD Reader 560 charges an internal power source 553 when connected to a docking station via recharging wires 554 that are electrically connected to contact rings 555, which are illustrated in FIG. 14B. In one aspect, an ultracapacitor based power source is used due to its long life.

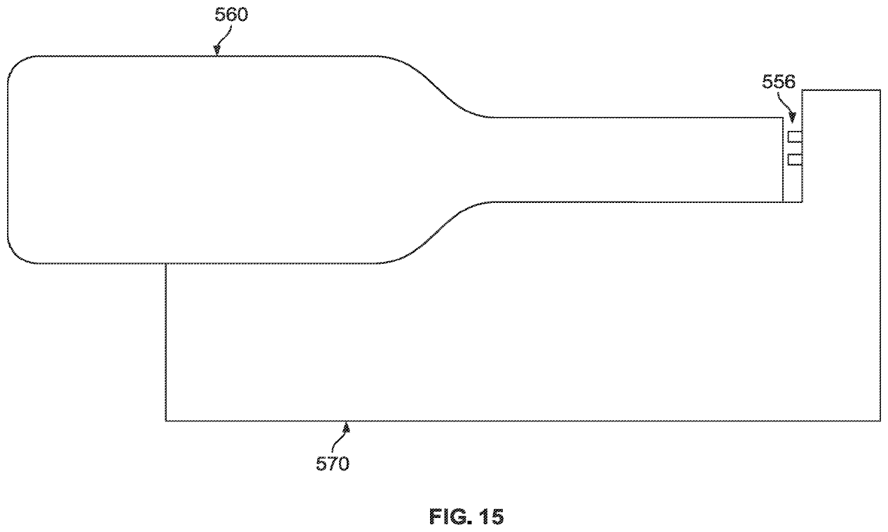

FIG. 15 illustrates the RTD Reader 560 received by a docking station 570. The charging contacts 556 provide power to the mobile power source 553 illustrated in FIG. 553. The capacity of the mobile power source 553 may be low because the RTD reader will only need battery power for a brief period while away from its docking station 570. In addition to charging, the RTD reader may communicate wirelessly or in wired communication with a controlling microprocessor over the serial communication line. The RTD may receive the following commands from the controlling microprocessor, which are described by way of example and not by limitation: i) set a real time clock; ii) clear the RTD reading memory; iii) upload the RTD reading memory; and iv) sense the connection of an RTD to the three RTD lines in the slip connector.

The RTD reader assembly 560 in FIG. 14A may be combined with calibrator bottle 130 illustrated in FIG. 3. Such combination may the number of bottle receptacle stations that are filled with non-patient samples, thereby freeing up more receptacles in the rack to receive sample containers. This in turn increases the capacity of the instrument to process even more samples. The calibrator bottle and combined wireless RTD reader bottle may be placed in column 0 in the drum assembly as described above. Alternatively, if the RTD reader assembly is designed as a separate component from the calibration bottle assembly, then one or more RTD assemblies may be placed into drum receptacles at a location of interest. The robotic arm assembly, which is typically responsible for inserting and removing patient sample bottles in the drum assembly may be used to move RTD bottle assemblies back to the docking station(s).

The reader cooperates with a memory to read a temperature for any connected RTD and storing the time stamped RTD readings. The memory may have any capacity.

A single RTD Reader will service up to 4 different blood culture modules. Each module has its own housing and incubation and reading environment. Additional data storage capacity is included to prevent overwriting of current values if multiple readings of a single RTD occur.

The RTD data storage may be a circular buffer with each new reading overwriting the oldest stored reading. However, RTD data storage is a matter of design choice. One of ordinary skill may select an RTD data storage compatible with its operational objective. The system described herein is not limited to any particular type of data storage.

In one aspect, the system described herein may include a Command Center The Command Center may track the time it commands the robot to move the RTD Reader to a reference bottle position. The Command Center may also manage workflows, robotic movements, etc. The Command Center is configured to correlate the time a specific RTD is read with the timestamp stored by the RTD Reader with the RTD reading. The controller may then detect when an RTD was not detected and read by the RTD Reader, and prevent the mis-association of RTD temperatures with the incorrect reference bottle position.

As noted above, the RTD device may have a cap portion configured for easy interconnection with the slip connector and other components that are interconnected with the RTD device to receive data therefrom. Described above are ring connectors that easily electrically engage the RTD device with other devices in the instrument for measurement and control of the system.

In one aspect, the RTD Reader is oriented so that it may engage electrically with the electrical connectors on the reference bottle holders and docking station.

The RTD reader docking station will have a form factor similar to that of a bottle holder in the drum-shaped rack. The RTD reader docking station will need access to power and a serial interface. Referring to FIG. 15, the form factor of the docking station is configured to receive a specific calibrator device configuration. Although not specifically illustrated, the illustrated docking station 570 may include a clip, a tray, or other structures to removably retain the calibration device in the docking station. Such structures are well known and are described in WO 2021/026272, which was published on Feb. 11, 2021 and is incorporated by reference herein.

In one aspect, a robot is used to insert and remove the RTD Reader from the docking station. For efficiency, the robot may be able to use the same motions for inserting and removing the RTD Reader from the docking station that it will use to insert and remove a bottle from the drum-shaped rack.

As described above, the docking station may have a slip connector, which is similar to the slip connector for the RTD device on the reference bottle holders described above. Such a slip connector may be mounted horizontally inside the bottle holder of the docking station. The slip connector on a USB Reader mates with this connector when the USB Reader is inserted into the docking station. In one aspect, the RTD Reader may be shaped to wrap around the light pipe 417 so the force holding it against the stop also orients it rotationally.

In one aspect, the reference bottle temperatures are collected based on instructions from the controller to stop the drum-shaped rack in a position where the reference bottle column is accessible, e.g., by a robotic mechanism that loads and unloads bottles from the rack.

The controller causes the door in the housing to open and sets the time in the RTD Reader via a microprocessor or other control device in communication with the command center for the instrument. Such control components are well known to the skilled person and not described in detail herein.

The robot then picks up the RTD Reader and moves to the drum-shaped rack. At each row of the drum, the robot moves the RTD Reader into contact with the slip connector for the RTD receptacle in that row.

The RTD Reader senses contact with the slip connector, measures the temperature from the RTD, time stamps the temperature data, and stores it in the RTD Reading buffer. The controller saves the time the row temperature was collected and associates it with the module and the row the robot is accessing.

Once the temperatures from all rows in the drum-shaped rack of the module are collected, the controller then commences to collect temperatures from the RTDs in another module. In the current module for which temperature collection is completed, the robot is moved to the RTD Reader docking station. The robot inserts the RTD Reader into the docking station, after which the robot may perform other tasks in the module (i.e., loading and unloading sample bottles).

The controller may call up the temperature data from the RTD Reader from the microprocessor or other control device described above. In response, the RTD Reader transfers all timestamped temperature readings to the controller via the VIS. The controller analyzes the temperature data for each module and determines if the incubation offset for that module needs to be updated. If an update is needed, the new incubation offset is sent to the module.

### Adjusting the Incubation Offset

In one aspect, the temperature measured from the reference bottles may not be used directly for the control of the air temperature in the incubation chamber. The reference bottle temperature probe, being located in the liquid in the reference bottle, may not respond fast enough for proper air temperature control. The reference bottle temperatures may be used to periodically adjust the offset used for the air temperature control.

In one aspect, the module may determine its incubation offsets for each incubation chamber during final testing after manufacture. The incubation offset is most affected by the ambient temperature at which the instrument is operating, so the initial incubation offset would be for the ambient conditions experienced during manufacturing. Each incubation system may store and use its individual incubation offset.

For any specific ambient condition, the incubation offset may remain unchanged, so any adjustment of the incubation offset occurs slowly. The reference bottle temperatures may be read and averaged regularly (e.g., once an hour, or once a day, etc.) and the incubation offset only allowed to change by some small increment at each update. This protocol avoids substantial changes in the offset and hence substantial changes in the bottle temperatures, which may adversely affect the fluorescent readings measured by the measurement system.

In one aspect, the module instrument is operated at a steady state for some period of time (to achieve, e.g., stable heater output) before the reference bottle temperatures may be used to update the incubation offset. The heater output is monitored continuously, and changes to the incubation offset may not be needed until a significant change occurs in the heater output, indicating a change to the ambient temperature.

### Charging the RTD Reader

As explained above, the RTD reader is charged whenever it is in its docking station via the slip connectors. In one aspect the power source in the RTD reader is configured to have a long life and be replaceable by the user when necessary. In other words, the RTD reader is a critical tool for the module and must be maintained in good and reliable operational order.

In one aspect, the incubation offset is selected during development (i.e. pre-manufacture) of the incubation system by measuring the temperature of the liquid in the media bottles during operation of a module at the extremes of the incubation temperatures that will be used during system operation. The incubation offset is selected such that the full range of bottle media temperatures are within the temperature specification for all ambient temperature conditions. Collecting the temperature of the media bottle liquid contents during operation requires special equipment that may only be used during development of the instrument.

As stated above. the incubation offset established during development of the instrument remains virtually constant for all instruments subsequently made, for the life of the product, operating in all conditions, except for the minor incremental changes in the offset described above. However, if the RTD devices are deployed in the module, then access to reference bottle temperatures in the drum during module operation is possible. This allows the module to determine the incubation offset directly and allows the incubation system to account for the actual ambient environment of the incubation system. Such RTD devices permit the system to control the temperatures of the blood culture bottles more accurately in the incubation chamber.

Ambient temperature control is more challenging at lower temperatures than at higher temperatures. At low ambient temperatures, heat is lost from the incubation chamber more rapidly than at high ambient temperatures. Consequently, the average bottle temperature tends to be lower when the instrument is operating at a low ambient temperature because the average air temperature in the module is lower. When the incubation offset is determined during development, there is a tradeoff between the average bottle temperatures at low ambient temperature and high ambient temperature, since the offsets may be different at the upper and lower portions of the ambient temperature range. When the RTD devices described herein are deployed, the actual average of the bottle temperatures could be calculated at an ambient temperature and the incubation offset adjusted appropriately for a specific ambient temperature.

When the RTD devices described herein are deployed, the module is provided with a tool to adjust the incubation offset during operation of the module. Therefore, universal offset is not required to be designed in to the module. The design may be to provide a range of temperatures for the incubated bottles, with the offset being determined as the module is operated. As stated previously, the rough control of the bottle temperature is the air flow and the air path in the module and temperature of the heated air in the module. The average temperature of the incubated bottles is controlled by the change of the air temperature as it moves through the incubation chamber. The RTD provides the fine control to obtain more precision in heating the bottle contents to a set temperature, without relying on a universal offset that might not achieve the set temperature in every circumstance.

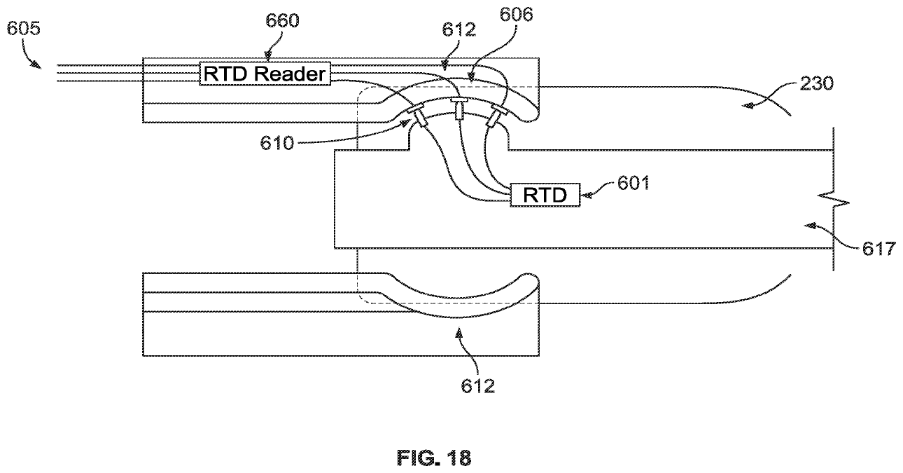

Referring to FIG. 18, in a different aspect, instead of using an RTD Reader to collect temperatures from the reference bottles and transmit them to the microprocessor in communication with the command center describe above, the RTD Reader 660 may be a connector that includes power, ground and serial communication lines 605 and contacts 606 included in a gripper finger 612. The RTD Reader 660 electronics may be provided in the gripper fingers 612 of a robot used to grasp and release the bottles 230 as they are inserted into and removed from the bottle rack in the module. The contacts 606 may connect to the contacts 610 of the RTD device 601 disposed in a reference bottle holder (e.g., the light pipe 617) when the robot 612 grips the reference bottle. The electronics may be connected through an interface so that the measured temperatures are recorded and used as described above.

In one aspect, the connectors 606 on the robot fingers may be spring loaded pins (e.g., pogo pins) that mate with corresponding contacts 610 on the light pipe 617 of the reference bottle holder 617. As described above, in one aspect there are three contacts 610 for the connection to the RTD. The connector may be mounted to a small board, which would be mounted to the outside surface of one of the gripper fingers 612. The gripper finger 612 containing the RTD Reader 660 may be shaped such that the board is recessed into the finger 612, reducing the chance for interference with objects the gripper 612 must move around. In a further aspect, the small board that contains the RTD connection 610 also houses electronics to read the RTD in the light pipe 617. When the electronics are placed on this board there is no need to run the RTD analog signals through longer wires that run through the robot wire guides 605 to the microprocessor that receives the temperature information for the RTD device. This increases the accuracy of the RTD readings.

### Representative Workflow:

A controller (referred to herein as a Command Center) requests for the module to stop rotation of its drum-shaped rack with the reference bottle column accessible by the bottle robot. The Command Center then causes a module hatch to open for the robot to access the bottles disposed in the rack. The Command Center then controls the movement of the robot to the drum. At each row of the drum, the Command Center instructs the robot to grip the reference bottle for that row. The Command Center then confirms that the necessary connects are made such that the RTD electronics are connected to collect the temperature from the currently connected RTD. After measurement is confirmed, the Command Center instructs the robot to release the reference bottle. Once the temperatures from the references bottles in all rows in the module drum-shaped rack are collected, the robot is moved away from the drum, after which the Command Center causes the module hatch to close. The Command Center then releases control of the drum-shaped rack of the module back to the module. The Command Center then releases the robot for other workflows. Following on, the Command Center analyzes the temperature data for each module and determines if the incubation offset for that module needs to be updated. If an update is needed, the new incubation offset is provided to the module.

Direct hardwire RTD connection to Command Center

In another aspect, the RTD temperatures measured from the reference bottles are collected periodically when the drum is stopped. In this aspect, the bottom of the drum has a multi-pin slip connector (several pads) that connect back to each reference bottle. In a further aspect, only temperatures from a few of the bottles in the drum-shaped rack column are collected. For example, in one aspect, temperatures from a top row, a bottom row, and a middle row are collected.

In one aspect, a set of pogo pins on the floor of the module may extend upward via a motorized slide rail (or similar mechanism to automate upward translation) to contact the pads disposed on the bottle shaped RTD device while the drum is in the stopped position. A wire harness may extend from the pogo pins, connecting them to a controller or microprocessor that records the temperature of the bottle contents and correlates the temperature with the rack position for the bottle associated with the temperature measurement. This data may be provided to the Command Center, which may then use it to determine a temperature offset.

Wireless RTD connection to Command Center

In a further aspect, the wiring and connectors describe hereinabove may be avoided by deploying a wireless connection. In this aspect, RTD data is transmitted using low power transmission hardware typical for popular wireless protocols such as IOT protocols, Bluetooth, WiFi, and RFID with EEPROM. The wireless interface device will have a power source. In one aspect the power source may be batteries mounted on a frame for the drum-shaped rack. Alternatively, a bottle shaped rechargeable battery may be provided that will provide power. Since a rechargeable battery requires recharging, the module would be configured be able to remove and replace the rechargeable battery. In one aspect, the robot described above removes and replaces the rechargeable battery. The module or system (in the case of multiple modules) may store additional rechargeable batteries for use as required.

The term “about,” as used herein indicates that there is some variability in the ranges and values expressed. One skilled in the art is aware of such variations and the extent of variability associated with each value. Typically, such variation will be not more than 25% of the value expressed and may be no more than 20%, no more, than 15%, no more than 10%, and no more than 5% of the value expressed.

Described herein is a calibration device made of a plastic material, wherein the plastic material is doped or compounded with or implanted with at least one dye or pigment or compound that has a fluorescent emission spectrum within a first predetermined wavelength range when excited by light of a second predetermined wavelength range. In one aspect the plastic material does not absorb light in either the first predetermined wavelength range or the second predetermined wavelength range. In another aspect, the calibration device is a bottle formed of the plastic material that may be polycarbonate. The calibration device may have an interrogation area formed as an aperture in the calibration device wherein the plastic material is inserted in the aperture of the calibration device. A neutral density filter may placed over the plastic material. In one aspect, the neutral density filter comprises a plurality of neutral density filters and the plurality of neutral density filters may be formed in a stack. In one aspect, the at least one dye or pigment or compound is provided as a fluorescent material system selected to provide the fluorescent emission spectrum and may be an organic dye.

Described herein is a method of calibrating an instrument for detecting sample fluorescence in which a calibration device having a plastic material, wherein the plastic material is doped or compounded with or implanted with at least one dye or pigment or compound that has a fluorescent emission spectrum within a first predetermined wavelength range when excited by light of a second predetermined wavelength range is provided and placed in an instrument with a sensor. The calibration device and the sensor are brought into alignment. Light is directed onto the calibration device from a light source, wherein the light source emits light in the second predetermined wavelength range to produce fluorescence in the first predetermined wavelength range. It is then determined if the sensor detects the fluorescence in the first predetermined wavelength range. If the sensor does not detect the fluorescence in the first predetermined wavelength range, the instrument is adjusted and re-calibrated by bringing into alignment the calibration device with the sensor. Again, a light source directs light onto the calibration device, wherein the light source emits light in the second predetermined wavelength range to produce fluorescence in the first predetermined wavelength range. It is then determined if the sensor detects the fluorescence in the first predetermined wavelength range. In one aspect, the plastic material does not absorb light in either the first predetermined wavelength range or the second predetermined wavelength range. In one aspect, the calibration device is a bottle formed of the plastic material that may be polycarbonate. In one aspect, the calibration device has an interrogation area formed as an aperture in the calibration device wherein the plastic material is inserted in the aperture of the calibration device. In one aspect, a neutral density filter is placed over the plastic material. The neutral density filter may be a plurality of neutral density filters that may be formed in a stack. In one aspect of the above method, the at least one dye or pigment or compound is provided as a fluorescent material system selected to provide the fluorescent emission spectrum, and the dye may be an organic dye.

Also described is an instrument for blood culture incubation, the instrument having a housing, a rack disposed in the housing the rack comprising a plurality of rack receptacles for receiving a plurality of blood culture sample containers. In one aspect, at least one rack receptacle is configured to receive a temperature measuring device. The temperature measuring device may have a resistance temperature detector and the instrument may be configured to read the temperature recorded by the resistance temperature detector and from that temperature infer the temperature of a blood culture in a sample container. In one aspect, the instrument may have a controller, and the controller, when receiving a temperature from the temperature measuring device, may compare such temperature to a set temperature, and, based upon the comparison, control the temperature in the housing. In a further aspect, the rack receptacles may have a light pipe and the receptacle may be configured to receive a temperature measuring device that is embedded in the light pipe. In a further aspect, the receptacle configured to receive a temperature measuring device has a thermal grease or a thermal conductive pad applied thereon. In another aspect, the resistance temperature detector is electrically coupled to a cable having a power wire, a ground wire, and a serial communication wire. In one aspect, the cable is coupled to a slip connector. In a further aspect of the instrument, the temperature measuring device may be container-shaped, wherein the shape is configured to be received by the receptacle configured to receive the temperature measuring device. In a further aspect, the resistance temperature detector is immersed in liquid inside the container-shaped measuring device and may be coupled to a cable with contacts that are configured to electrically connect to a slip connector adjacent to the receptacle configured to receive the temperature measuring device. In one aspect, the contacts are contact rings and the contacts may be placed in spaced relation on the surface of the temperature measuring device. In a further aspect of the instrument, the resistance temperature detector may be coupled to a cable comprising a contact strip, with contacts that are configured to electrically connect to corresponding spring contacts in electrical communication with a microprocessor. In one aspect, the temperature measuring device may have a transmitter and a power source. In one aspect, the temperature measuring device may be rechargeable. In a further aspect of the instrument, the container-shaped temperature measuring device is formed of a plastic material, and the plastic material may be doped or compounded with or implanted with at least one dye or pigment or compound that has a fluorescent emission spectrum within a first predetermined wavelength range when excited by light of a second predetermined wavelength range. In any of the above aspects, the container-shaped temperature measuring device may be a bottle shape. In a further aspect, the plastic material may not absorb light in either the first predetermined wavelength range or the second predetermined wavelength range and may be polycarbonate. The plastic material may be doped or compounded with or implanted with at least one dye or pigment or compound and that is provided as an insert in an aperture formed in the container-shaped temperature measuring device. In one aspect, the container-shaped measuring device may be formed from the plastic material that is doped or compounded with or implanted with at least one dye or pigment or compound.

From the foregoing and with reference to the various figure drawings, those skilled in the art will appreciate that certain modifications may also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A calibration device comprising:
a bottle formed of a plastic material, wherein the plastic material is doped or compounded with or implanted with at least one dye or pigment or compound that has a fluorescent emission spectrum within a first predetermined wavelength range when excited by light of a second predetermined wavelength range; and wherein the plastic material does not absorb light in either the first predetermined wavelength range or the second predetermined wavelength range.

2. (canceled)

3. (canceled)

4. (canceled)

5. The calibration device of claim 1, wherein the calibration device comprises an interrogation area formed as an aperture in the calibration device wherein the plastic material is inserted in the aperture of the calibration device.

6. The calibration device of claim 5, wherein a neutral density filter is placed over the plastic material.

7. The calibration device of claim 6, wherein the neutral density filter comprises a plurality of neutral density filters.

8. The calibration device of claim 7, wherein the plurality of neutral density filters is formed in a stack.

9. The calibration device of claim 8, wherein the plastic material is polycarbonate.

10. The calibration device of claim 1, wherein the at least one dye or pigment or compound is provided as a fluorescent material system selected to provide the fluorescent emission spectrum, and wherein the dye is an organic dye.

11. (canceled)

12. A method of calibrating an instrument for detecting sample fluorescence comprising:
providing a calibration device that is a bottle formed of a plastic material, wherein the plastic material is doped or compounded with or implanted with at least one dye or pigment or compound that has a fluorescent emission spectrum within a first predetermined wavelength range when excited by light of a second predetermined wavelength range;
placing the calibration device in an instrument with a sensor;
bringing into alignment the calibration device with the sensor;
directing light from a light source, wherein the light source emits light in the second predetermined wavelength range to produce fluorescence in the first predetermined wavelength range; and
determining if the sensor detects the fluorescence in the first predetermined wavelength range.

13. The method of claim 12, wherein, if the sensor does not detect the fluorescence in the first predetermined wavelength range, the method further comprises adjusting the instrument and re-calibrating the instrument by bringing into alignment the calibration device with the sensor;
directing light from a light source, wherein the light source emits light in the second predetermined wavelength range to produce fluorescence in the first predetermined wavelength range; and
determining if the sensor detects the fluorescence in the first predetermined wavelength range.

14. The method of claim 12, wherein the plastic material does not absorb light in either the first predetermined wavelength range or the second predetermined wavelength range.

15. (canceled)

16. The method of claim 12, wherein the plastic material is polycarbonate and wherein the calibration device comprises an interrogation area formed as an aperture in the calibration device wherein the plastic material is inserted in the aperture of the calibration device.

17. (canceled)

18. The method of claim 16, wherein a neutral density filter is placed over the plastic material.

19. The method of claim 18, wherein the neutral density filter comprises a plurality of neutral density filters.

20. The method of claim 19, wherein the plurality of neutral density filters is formed in a stack.

21. (canceled)

22. The method of claim 12, wherein the at least one dye or pigment or compound is provided as a fluorescent material system selected to provide the fluorescent emission spectrum and wherein the dye is an organic dye.

23. (canceled)

24. An instrument for blood culture incubation, the instrument comprising:
a housing;
a rack disposed in the housing the rack comprising a plurality of rack receptacles for receiving a plurality of blood culture sample containers;
at least one rack receptacle configured to receive a temperature measuring device; and
wherein the temperature measuring device comprises a resistance temperature detector; and
wherein the instrument is configured to read the temperature recorded by the resistance temperature detector and from that temperature infer the temperature of a blood culture in a sample container.

25. The instrument of claim 24, wherein the instrument further comprises a controller, wherein the controller, when receiving a temperature from the temperature measuring device, compares such temperature to a set temperature, and, based upon the comparison, controls the temperature in the housing.

26. The instrument of claim 24, wherein the rack receptacles comprise a light pipe and the receptacle configured to receive a temperature measuring device has the temperature embedded in the light pipe, wherein the receptacle configured to receive a temperature measuring device has a thermal grease or a thermal conductive pad applied thereon.

27. (canceled)

28. The instrument of claim 26, wherein the resistance temperature detector is electrically coupled to a cable comprising a power wire, a ground wire, and a serial communication wire, and wherein the cable is coupled to a slip connector.

29. (canceled)

30. The instrument of claim 24, wherein the temperature measuring device is container-shaped, wherein the shape is configured to be received by the receptacle configured to receive the temperature measuring device, wherein the container-shaped temperature measuring device has a bottle shape; and wherein the container-shaped temperature measuring device comprises a plastic material, wherein the plastic material is doped or compounded with or implanted with at least one dye or pigment or compound that has a fluorescent emission spectrum within a first predetermined wavelength range when excited by light of a second predetermined wavelength range; and wherein the resistance temperature detector is immersed in liquid inside the container-shaped measuring device.

31. (canceled)

32. The instrument of claim 30, wherein the resistance temperature detector is coupled to a cable with contacts that are configured to electrically connect to a slip connector adjacent to the receptacle configured to receive the temperature measuring device, and wherein the contacts are contact rings.

33. (canceled)

34. The instrument of claim 32, wherein the contacts are placed in spaced relation on a surface of the temperature measuring device.

35. The instrument of claim 24, wherein the resistance temperature detector is coupled to a cable comprising a contact strip, with contacts that are configured to electrically connect to corresponding spring contacts in electrical communication with a microprocessor.

36. The instrument of claim 30, wherein the temperature measuring device further comprises a transmitter and a power source and wherein the temperature measuring device is rechargeable.

37. (canceled)

38. (canceled)

39. (canceled)

40. The instrument of claim 30, wherein the plastic material does not absorb light in either the first predetermined wavelength range or the second predetermined wavelength range and wherein the plastic material comprises polycarbonate.

41. (canceled)

42. The instrument of claim 40, wherein the plastic material that is doped or compounded with or implanted with at least one dye or pigment or compound is provided as an insert in an aperture formed in the container-shaped temperature measuring device or wherein the container-shaped measuring device is formed from the plastic material that is doped or compounded with or implanted with at least one dye or pigment or compound.

43. (canceled)
